(19) United States
(12) Patent Application Publication
(10) Pub. No.: US 2008/0146911 A1
(43) Pub. Date: Jun. 19, 2008

(54) Respiration Monitoring Apparatus, Respiration Monitoring System, Medical Processing System, Respiration Monitoring Method, And Respiration Monitoring Program

(75) Inventor: Yoshio Miyake, Tokyo (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignees: KABUSHIKI KAISHA TOSHIBA, Tokyo (JP); TOSHIBA SOLUTIONS CORPORATION, Tokyo (JP)

(21) Appl. No.: 11/665,685

(22) PCT Filed: Oct. 17, 2005
(86) PCT No.: PCT/JP05/19032

§ 371 Date: Apr. 17, 2007

(30) Foreign Application Priority Data
Oct. 18, 2004 (JP) 2004-302558

(51) Int. Cl. A61B 5/08; A61B 5/05

(52) U.S. Cl. 600/411; 600/529; 600/407

(57) ABSTRACT

A respiration monitoring apparatus comprises an image-acquiring unit that acquires images at predetermined timings, each image photographed in a direction inclined at a prescribed angle to a region-of-interest including at least one of a subject' s chest and abdomen; and an expiration/aspiration discriminating unit that determines a direction in which a pixel moves, from a positional change of the pixel in an image, on the basis of a plurality of images acquired at consecutive timings by the image-acquiring unit, and discriminates between the subject's expiration and aspiration on the basis of the direction determined.

### TECHNICAL FIELD

The present invention relates to a respiration monitoring process for discriminating between the subject's expiration and aspiration.

### BACKGROUND ART

Hitherto, a technique called respiration-synchronized scan is utilized in imaging processes such as CT scan. This scan is a technique of scanning organs that move as the subject breathes (e.g., lungs, liver, spleens, or the like), at a certain phase of the respiration cycle, thereby to obtain images of the organs.

This technique helps to provide clear images of any organ susceptible to motion artifact due to respiration, not influenced so much by the motion artifact.

In the conventional respiration-synchronized scan, a device is attached to the subject in most cases, which can detect a tension resulting from the respiration, thereby to discriminate between expiration and aspiration. (The device is attached to, for example, a part near the chest or the abdomen.)

The device is attached directly to the subject to discriminate expiration and aspiration, in the conventional technique. The device thus attached to the subject may give the subject discomfort or may come into the view field of the imaging apparatus.

### DISCLOSURE OF INVENTION

### Problems to be Solved by the Invention

The present invention has been made to solve the problems described above. An object of the invention is to provide a technique that can discriminate between the subject's expiration and aspiration, without contacting the subject.

### Means for Solving the Problems

To solve the above-mentioned problems, a respiration monitoring apparatus according to the present invention comprises: an image-acquiring unit that acquires images at predetermined timings, each image photographed in a direction inclined at a prescribed angle to a region-of-interest including at least one of a subject's chest and abdomen; and an expiration/aspiration discriminating unit that determines a direction in which a pixel moves, from a positional change of the pixel in an image, on the basis of a plurality of images acquired at consecutive timings by the image-acquiring unit, and discriminates between the subject's expiration and aspiration on the basis of the direction determined.

In the respiration monitoring apparatus configured as described above, the expiration/aspiration discriminating unit may determine that the subject is inhaling when the pixel in the image moves in a first direction that is a direction component in a plane substantially parallel to longitudinal and transverse directions of the subject, and that the subject is exhaling when the pixel in the image moves in a second direction that is opposite to the first direction.

In the respiration monitoring apparatus configured as described above, a speed dy/dt of all pixels in the region-of-interest may be given as follows with respect to the direction substantially parallel to the longitudinal direction of the subject:

$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$

where y is an ordinate on the image acquired by the image-acquiring unit with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y) at the time t, the first Σ is the sum for all pixels with respect to one of the x and y directions in the region-of-interest, and the second Σ is the sum for all pixels with respect to the other of the x and y directions in the region-of-interest. The expiration/aspiration discriminating unit may determine that the subject is inhaling when the pixel moves at the speed dy/dt in the first direction and that the subject is exhaling when the pixel moves at speed dy/dt in the second direction.

In the respiration monitoring apparatus configured as described above, the expiration/aspiration discriminating unit may extract a second region of an image acquired at a prescribed timing and having pixels of almost the same luminance distribution as those of a first region composed of given pixels existing in the image acquired at the timing immediately preceding that prescribed timing, and may determine that the subject is inhaling when the component of the direction in which the pixel moves from the first region to the second region is oriented in the first direction and that the subject is exhaling when the component of the direction is oriented in the second direction.

Preferably, the respiration monitoring apparatus configured as described above may further comprise: a respiration-cycle determining unit that determines a respiration cycle of the subject, on the basis of images acquired at the predetermined consecutive timings by the image-acquiring unit and from the change in the luminance of pixels in the images, which occurs with time; and an expiration/aspiration timing determining unit that determines the timing of the expiration or aspiration discriminated by the expiration/aspiration discriminating unit, on the basis of the respiration cycle determined by the respiration-cycle determining unit.

A respiration monitoring apparatus according to the present invention comprises: an image-acquiring unit that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and an expiration/aspiration discriminating unit that discriminates between the subject's expiration and aspiration, on the basis of a plurality of images acquired at the predetermined timings by the image-acquiring unit and in accordance with whether a part of the region-of-interest, which is composed of pixels having luminance higher than a prescribed value, increases or decreases in area.

In the respiration monitoring apparatus configured as described above, the expiration/aspiration discriminating unit may determine that the subject is inhaling when that part of the region-of-interest, which is composed of pixels having luminance higher than a prescribed value, increases in area, and that subject is exhaling when that part of the region-of-interest, which is composed of pixels having luminance higher than a prescribed value, decreases in area.

The respiration monitoring apparatus configured as described above may further comprise a respiration-cycle determining unit that determines a respiration cycle of the subject, on the basis of images acquired at the predetermined consecutive timings by the image-acquiring unit and from the change in the luminance of pixels in the images, which occurs with time; and an expiration/aspiration timing determining unit that determines the timing of the expiration or aspiration discriminated by the expiration/aspiration discriminating unit, on the basis of the respiration cycle determined by the respiration-cycle determining unit.

Preferably, the respiration monitoring apparatus configured as described above may further comprise a notification unit that accumulates the absolute values of the luminance changes of the pixels in the images acquired by the image-acquiring unit at predetermined consecutive timings, as long as the expiration/aspiration discriminating unit determines that the subject is inhaling, and notifies that the subject is inhaling, when a difference between the value thus accumulated and a predetermined value exceeds a preset value.

A respiration monitoring system according to this invention comprises: a respiration monitoring apparatus of such a type as described above; and an imaging unit that is located above the feet of the subject lying on the back and photographs the region-of-interest in a direction slantwise with respect to the region-of-interest.

A respiration monitoring system according to this invention comprises: a respiration monitoring apparatus of such a type as described above; and an imaging unit that photographs, sideways with respect to a subject, a region-of-interest including at least one of the subject's chest and abdomen and a boundary between at least one of the chest and abdomen and a background having lower illuminance than at least one of the chest and abdomen.

A medical processing system according to this invention has: a respiration monitoring apparatus of such a type as described above; and a medical-process executing unit that performs a predetermined medical process at a timing of the expiration or aspiration determined by the medical-process executing unit.

In the medical processing system as described above, the predetermined medical process may preferably be an imaging process performed by MRI. Nonetheless, the process may be an imaging process performed by CT scan.

A respiration monitoring method according to the present invention comprises: an image-acquiring step that acquires images at predetermined timings, each image photographed in a direction inclined at a prescribed angle to a region-of-interest including at least one of a subject's chest and abdomen; and an expiration/aspiration discriminating step that determines a direction in which a pixel moves, from a positional change of the pixel in an image, on the basis of a plurality of images acquired at consecutive timings in the image-acquiring step, and discriminates between the subject's expiration and aspiration on the basis of the direction determined.

In the respiration monitoring method configured as described above, it is desirable that, in the expiration/aspiration discriminating step, the subject is determined to be inhaling when the pixel in the image moves in a first direction that is a direction component in a plane substantially parallel to longitudinal and transverse directions of the subject, and is determined to be exhaling when the pixel in the image moves in a second direction that is opposite to the first direction.

In the respiration monitoring method configured as described above, a speed dy/dt of all pixels in the region-of-interest may be given as follows with respect to the direction substantially parallel to the longitudinal direction of the subject:

$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$

where y is an ordinate on the image acquired by the image-acquiring step with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y), the first Σ is the sum for all pixels with respect to one of the x and y directions in the region-of-interest, and the second Σ is the sum for all pixels with respect to the other of the x and y directions in the region-of-interest. Further, in the expiration/aspiration discriminating step, the subject may be determined to be inhaling when the pixel moves at the speed dy/dt in the first direction and may be determined to be exhaling when the pixel moves at speed dy/dt in the second direction.

In the respiration monitoring method configured as described above, it is desired that, in the expiration/aspiration discriminating step, a second region of an image acquired at a prescribed timing and having pixels of almost the same luminance distribution as those of a first region composed of given pixels existing in the image acquired at the timing immediately preceding that prescribed timing is extracted, and the subject is determined to be inhaling when the component of the direction in which the pixel moves from the first region to the second region is oriented in the first direction and to be exhaling when the component of the direction is oriented in the second direction.

The respiration monitoring method configured as described above may further comprise: a respiration-cycle determining step that determines a respiration cycle of the subject, on the basis of images acquired at the predetermined consecutive timings in the image-acquiring step and from the change in the luminance of pixels in the images, which occurs with time; and an expiration/aspiration timing determining step that determines the timing of the expiration or aspiration discriminated in the expiration/aspiration discriminating step, on the basis of the respiration cycle determined in the respiration-cycle determining step.

Preferably, a respiration monitoring method according to this invention may comprise: an image-acquiring step that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and an expiration/aspiration discriminating step that discriminates between the subject's expiration and aspiration, on the basis of a plurality of images acquired at the predetermined timings in the image-acquiring step and in accordance with whether a part of the region-of-interest, which is composed of pixels having luminance higher than a prescribed value, increases or decreases in area.

In the respiration monitoring method configured as described above, in the expiration/aspiration discriminating step, the subject may be determined to be inhaling when that part of the region-of-interest, which is composed of pixels having luminance higher than a prescribed value, increases in area, and the subject may be determined to be exhaling when that part of the region-of-interest, which is composed of pixels having luminance higher than a prescribed value, decreases in area.

Preferably, the respiration monitoring method configured as described above may have: a respiration-cycle determining step that determines a respiration cycle of the subject, on the basis of images acquired at the predetermined consecutive timings in the image-acquiring step and from the change in the luminance of pixels in the images, which occurs with time; and an expiration/aspiration timing determining step that determines the timing of the expiration or aspiration discriminated in the expiration/aspiration discriminating step, on the basis of the respiration cycle determined in the respiration-cycle determining step.

Preferably, the respiration monitoring method configured as described above may have: a notification step that accumulates the absolute values of the luminance changes of the pixels in the images acquired in the image-acquiring step at predetermined consecutive timings, as long as the subject is determined to be inhaling in the expiration/aspiration discriminating step, and notifying that the subject is inhaling, when a difference between the value thus accumulated and a predetermined value exceeds a preset value.

The respiration monitoring method configured as described above may have a medical-process executing step that performs a predetermined medical process at a timing of the expiration or aspiration determined in the expiration/aspiration timing determining step.

In the respiration monitoring method configured as described above, the predetermined medical process may preferably be an imaging process performed by MRI. Nonetheless, the process maybe an imaging process performed by CT scan.

A respiration monitoring program according to this invention enables a computer to perform: an image-acquiring step that acquires images at predetermined timings, each image photographed in a direction inclined at a prescribed angle to a region-of-interest including at least one of a subject's chest and abdomen; and an expiration/aspiration discriminating step that determines a direction in which a pixel moves, from a positional change of the pixel in an image, on the basis of a plurality of images acquired at consecutive timings in the image-acquiring step, and discriminates between the subject's expiration and aspiration on the basis of the direction determined.

In the respiration monitoring program configured as described above, in the expiration/aspiration discriminating step, the subject may be determined to be inhaling when the pixel in the image moves in a first direction that is a direction component in a plane substantially parallel to longitudinal and transverse directions of the subject, and may be determined to be exhaling when the pixel in the image moves in a second direction that is opposite to the first direction.

In the respiration monitoring program configured as described above, a speed dy/dt of all pixels in the region-of-interest is given as follows with respect to the direction substantially parallel to the longitudinal direction of the subject:

$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$

where y is an ordinate on the image acquired by the image-acquiring step with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y), the first Σ is the sum for all pixels with respect to one of the x and y directions in the region-of-interest, and the second Σ is the sum for all pixels with respect to the other of the x and y directions in the region-of-interest. In the expiration/aspiration discriminating step, the subject is determined to be inhaling when the pixel moves at the speed dy/dt in the first direction and is determined to be exhaling when the pixel moves at speed dy/dt in the second direction.

The respiration monitoring program configured as described above may preferably be so described that, in the expiration/aspiration discriminating step, a second region of an image acquired at a prescribed timing and having pixels of almost the same luminance distribution as those of a first region composed of given pixels existing in the image acquired at the timing immediately preceding that prescribed timing is extracted, and the subject is determined to be inhaling when the component of the direction in which the pixel moves from the first region to the second region is oriented in the first direction and to be exhaling when the component of the direction is oriented in the second direction.

The respiration monitoring program configured as described above may further have: a respiration-cycle determining step that determines a respiration cycle of the subject, on the basis of images acquired at-the predetermined consecutive timings in the image-acquiring step and from the change in the luminance of pixels in the images, which occurs with time; and an expiration/aspiration timing determining step that determines the timing of the expiration or aspiration discriminated in the expiration/aspiration discriminating step, on the basis of the respiration cycle determined in the respiration-cycle determining step.

A respiration monitoring program according to this invention enables a computer to perform: an image-acquiring step that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and an expiration/aspiration discriminating step that discriminates between the subject's expiration and aspiration, on the basis of a plurality of images acquired at the predetermined timings in the image-acquiring step and in accordance with whether a part of the region-of-interest, which is composed of pixels having luminance higher than a prescribed value, increases or decreases in area.

Preferably, the respiration monitoring program configured as described above may be so described that, in the expiration/aspiration discriminating step, the subject is determined to be inhaling when that part of the region-of-interest, which is composed of pixels having luminance higher than a prescribed value, increases in area, and the subject is determined to be exhaling when that part of the region-of-interest, which is composed of pixels having luminance higher than a prescribed value, decreases in area.

Preferably, the respiration monitoring program configured as described above may have: a respiration-cycle determining step that determines a respiration cycle of the subject, on the basis of images acquired at the predetermined consecutive timings in the image-acquiring step and from the change in the luminance of pixels in the images, which occurs with time; and an expiration/aspiration timing determining step that determines the timing of the expiration or aspiration discriminated in the expiration/aspiration discriminating step, on the basis of the respiration cycle determined in the respiration-cycle determining step.

The respiration monitoring program configured as described above may have: a notification step that accumulates the absolute values of the luminance changes of the pixels in the images acquired in the image-acquiring step at predetermined consecutive timings, as long as the subject is determined to be inhaling in the expiration/aspiration discriminating step, and notifies that the subject is inhaling, when a difference between the value thus accumulated and a predetermined value exceeds a preset value.

The respiration monitoring program configured as described above may have: a medical-process executing step that performs a predetermined medical process at a timing of the expiration or aspiration determined in the medical-process executing step.

In the respiration monitoring program configured as described above, the predetermined medical process may preferably be an imaging process performed by MRI. Nonetheless, the process maybe an imaging process performed by CT scan.

### BRIEF DESCRIPTION OF DRAWINGS

FIG. 1 is a function block diagram explaining a respiration monitoring apparatus, a respiration monitoring system and a medical processing system, all according to an embodiment of this invention;

FIG. 2 is a diagram representing a relation between the installation position of the imaging unit 101 and the motion of a pixel in the region-of-interest ROI, which occurs as the subject's chest or abdomen moves up and down due to the respiration;

FIG. 3 is another diagram representing a relation between the installation position of the imaging unit 101 and the motion of a pixel in the ROI, which occurs as the subject's chest or abdomen moves up and down because of the respiration;

FIG. 4 is a flowchart explaining the entire sequence of a respiration monitoring method performed in the embodiment of this invention;

FIG. 5 is a flowchart explaining, in detail, the difference process (S103) shown in FIG. 4;

FIG. 6 is a flowchart explaining a method of determining how a pixel moves in an image, by using the expiration/aspiration discriminating unit in the embodiment;

FIG. 7 is another flowchart explaining a method of determining how a pixel moves in an image, by using the expiration/aspiration discriminating unit in the embodiment;

FIG. 8 is a diagram showing the motion of a specific block on the screen and explaining a method of matching the block;

FIG. 9 is a diagram explaining a configuration that makes the imaging unit 101 scan the region to photograph, including the chest or abdomen of subject M, or both, sideways with respect the body of subject M;

FIG. 10 is another diagram explaining a configuration that makes the imaging unit 101 scan the region to photograph, including the chest or abdomen of subject M, or both, sideways with respect the body of subject M; and

FIG. 11 is a flowchart showing the entire sequence of a process performed by using a medical processing system that includes a respiration monitoring apparatus according to the embodiment.

### BEST MODE FOR CARRYING OUT THE INVENTION

### First Embodiment

The first embodiment of this invention will be described, with reference to the accompanying drawings.

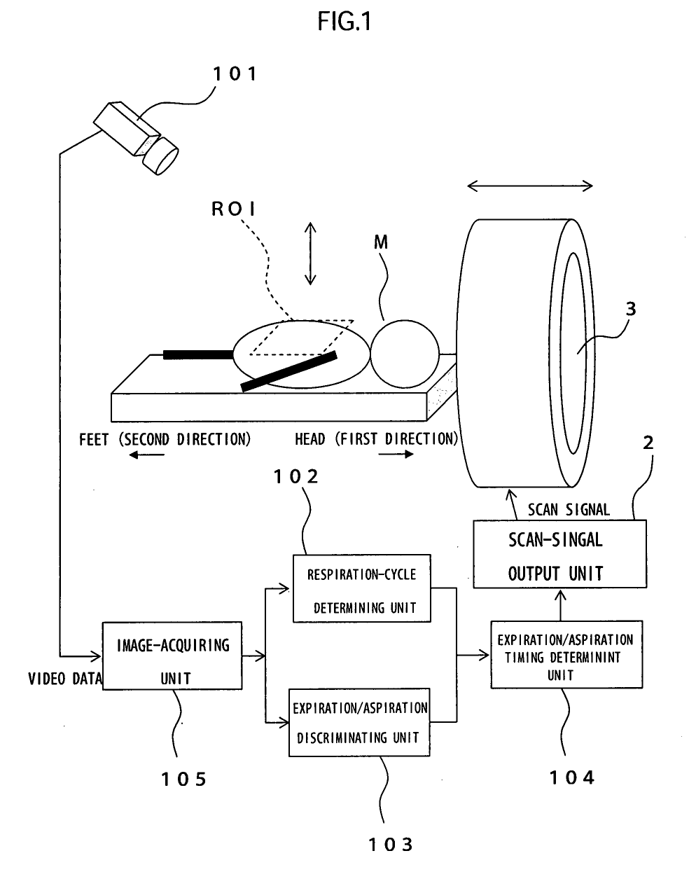

FIG. 1 is a function block diagram explaining a respiration monitoring apparatus, a respiration monitoring system and a medical processing system, all according to the present embodiment.

The respiration monitoring apparatus according to the present embodiment comprises a respiration-cycle determining unit 102, an expiration/aspiration discriminating unit 103, an expiration/aspiration timing determining unit 104, an image-acquiring unit 105, a storage unit (not shown), a control unit (not shown), a display unit (not shown), and a notification unit (not shown). The control unit is composed of a CPU, an image-processing circuit (image-processing chip), and the like. The medical processing system according to this embodiment comprises, in addition to the respiration monitoring apparatus, a scan-signal output unit (equivalent to a medical-process executing unit) 2, and a CT-scan apparatus 3. The respiration monitoring system according to this embodiment comprises the respiration monitoring apparatus and an imaging unit 101. The imaging unit 101 is located above the feet of the subject lying on the back and is so positioned to photograph the region-of-interest from above and slantwise.

The imaging unit 101 comprises a CCD camera and the like. It has the function of photographing the region-of-interest ROI that includes the chest or abdomen of the subject M, or both the chest and the abdomen, in a direction inclined at a predetermined angle to the plane of the region-of-interest ROI. More specifically, as is shown in FIG. 1, the imaging unit 101 is located above the feet of the subject M and directed slantwise to the region-of-interest ROI, in order to photograph the region-of-interest ROI.

The image-acquiring unit 105 has the function of acquiring video data generated by the imaging unit 101, at predetermined time intervals.

The respiration-cycle determining unit (cycle determining unit) 102 has the function of determining the respiration cycle of the subject M on the basis of the change in the luminance of pixels, which occurs with time and is detected from the consecutive images obtained at the predetermined time intervals.

The expiration/aspiration discriminating unit 103 determines the direction in which the pixels move, on the basis of the luminance change of pixels, which occurs with time and is detected from the consecutive images obtained at the predetermined time intervals. From this direction thus determined, the unit 103 discriminates between the subject's expiration and aspiration. More precisely, the expiration/aspiration discriminating unit 103 determines that the subject M is inhaling if the pixels move in the first direction (in the image) in a plane substantially parallel to the longitudinal and transverse directions of the subject M, i.e., a direction in which the subject M is photographed. If the pixels move in the second direction (on the image) that is opposite to the first direction, the unit 103 determines that the subject M is exhaling. The phrase “plane substantially parallel to the longitudinal and transverse directions of the subject M” means a plane that is almost horizontal while the subject M is laying on the back as shown in FIG. 1.

The imaging unit 101 is located above the feet of the subject M lying on the back and photographs the region-of-interest ROI in a direction slantwise with respect to the region-of-interest ROI. The expiration/aspiration discriminating unit 103 therefore determines that the subject M is inhaling if the pixels move in the image toward the head of the subject M (in the first direction) and that the subject M is exhaling if the pixels move toward the feet of the subject M (in the second direction).

The expiration/aspiration timing determining unit 104 has the function of determining the timing of the expiration or aspiration discriminated by the expiration/aspiration discriminating unit 103, on the basis of the respiration cycle the respiration-cycle determining unit 102 has determined.

The scan-signal output unit (medical-process executing unit) 2 generates a scan signal on the basis of the timing of expiration or aspiration determined by the expiration/aspiration timing determining unit 104. The scan signal is supplied to the CT-scan apparatus 3, which performs CT scanning, which is a medical processing.

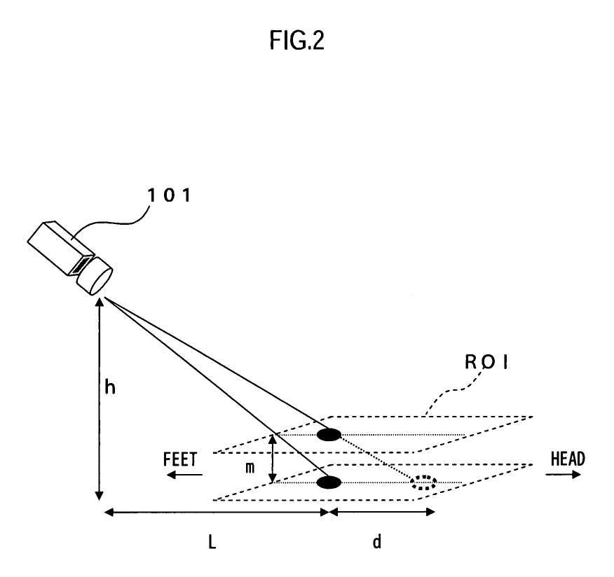

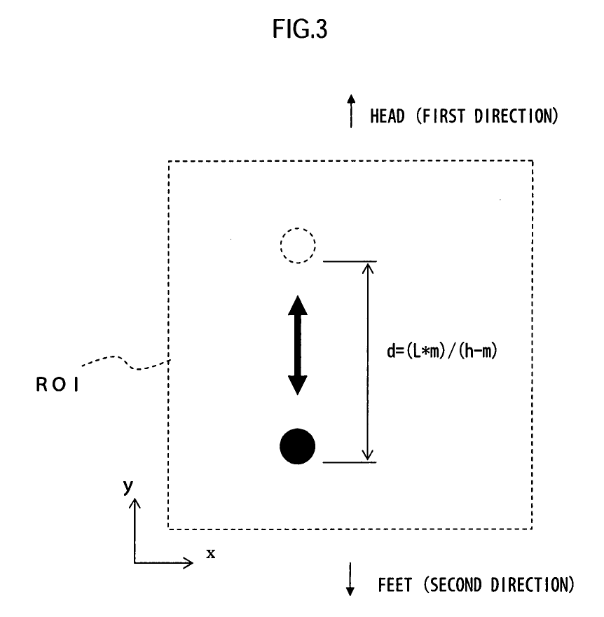

FIGS. 2 and 3 are diagrams representing a relation between the installation position of the imaging unit 101 and the motion of a pixel in the ROI, which occurs as the subject's chest or abdomen moves up and down because of the respiration.

As shown in FIGS. 2 and 3, a given point on the chest or abdomen of the subject M, which is the region-of-interest ROI photographed by the imaging unit 101, looks as if moving up and down in the image formed by the imaging unit 101 as the subject M breathes. Thus, the up-and-down movement of the subject's chest or abdomen can be determined from the positional changes of one pixel of the image formed by the imaging unit 101.

More specifically, distance d the pixel moves in the image formed by the imaging unit 101 is given as follows:

d=(L×m)/(h−m)

where h is the vertical distance between the imaging unit 101 and the given point on the chest or abdomen of the subject M, i.e., region-of-interest ROI, L is the horizontal distance between the imaging unit 101 and the given point on the chest or abdomen of the subject M, and m is the distance the region-of-interest ROI moves up and down as the subject M breathes.

To enable the CT-scan apparatus to perform its function continuously at a specific phase of the respiration cycle (that is, to achieve a respiration-synchronized scan), the phases of respiration must be determined, no matter how large or small the volume of respiration is.

In the present embodiment, the respiration-cycle determining unit 102 determines how the luminance of a pixel in the image of region-of-interest changes as the region is photographed at predetermined consecutive timings. (That is, the unit 102 calculates the difference in luminance between any two adjacent frames.) From the luminance changes of the pixel, the unit 102 determines the cycle of respiration.

To be more specific, the absolute real-time average change (difference) in luminance of the pixel in the image of the region-of-interest is obtained, and the absolute value of the difference is multiplied by the ratio of a fixed value to this average change. Waveforms the peak amplitude of which always approaches the fixed value is obtained (the difference is thus normalized). In other words, the timing of outputting the scan signal from the scan-signal output unit 2 can be designated on the basis of the waveforms thus processed.

The absolute value of the inter-frame difference (the luminance change of the pixel) between the image photographed at the latest timing and the image photographed at the timing immediately before the latest timing may be smaller than the absolute value of the inter-frame difference between the image photographed at the timing immediately before the latest timing and the image photographed at the timing immediately before this timing. Further, the absolute value of the luminance difference between several immediately preceding frames has increased. In this case, the timing immediately before the latest timing is regarded as the peak of the expiration/aspiration wave. Then, the time after a predetermined period from this peak may be used as timing of outputting the scan signal from the scan-signal output unit 2.

To accomplish the respiration-synchronized scan, it is necessary to determine which each wave represents, expiration or aspiration. As described above, expiration and aspiration can be discriminated, one from the other, in accordance with how the pixel moves in the image photographed. It will be explained how the expiration/aspiration discriminating unit 103 determines the direction the pixel moves in the image photographed by the imaging unit 101.

The pixel in the image of the region-of-interest moves to another position upon lapse of a predetermined short time (6t) as the subject breathes. Therefore, the following equation is established:

I(x,y,t)=I(x+δx,y+δy,t+δt)

where y is the ordinate on the image acquired by the image-acquiring unit 105 with respect to the longitudinal direction of the subject M, x is the abscissa on the image with respect to the direction intersecting at substantially right angles with the longitudinal direction of the subject M, t is time, and I(x,y,t) is the luminance of the pixel positioned at the coordinate (x,y).

Next, the right side of this equation is subjected to Tailor expansion, and the higher-order terms dx, dy and dt are neglected as being infinitesimal. The right term is then divided by dt. As a result:

(dx/dt)*∂I(x,y,t)/∂x+(dy/dt)*∂I(x,y,t)/∂y+∂I(x,y,t)/∂t=0

The changes in the speed of adjacent pixels, which take place at a certain time, can be considered almost the same. Hence, the error of the left term of the equation can be minimal for all adjacent pixels. Therefore:

E=ΣΣ((dx/dt)*∂I(x,y,t)/∂x+(dy/dt)*∂I(x,y,t)/∂y+∂I(x,y,t)/∂t)²

u=dx/dt, v=dy/dt

Then, the following two equations are established:

∂E/∂u=0, ∂E/∂v=0

From these equations, the speed dy/dt for all pixels is given:

$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix} \qquad ( 1 )$$

Here, of ΣΣ, the first Σ is the sum for all pixels with respect to one of the x and y directions in the region-of-interest, and the second Σ is the sum for all pixels with respect to the other of the x and y directions in the region-of-interest.

The solution to the equation (1), if obtained for the image of the region-of-interest ROI photographed, the distance and direction the image of the region (ROI) examined moves can be determined. If the region moves upwards (toward the head), it is determined that the subject is inhaling. If the region moves downwards (toward the feet), it is determined that the subject is exhaling. (That is, if dy/dt has a positive value, the image moves in the y direction toward the head, indicating that the subject is exhaling, whereas if the dy/dt has a negative value, the image moves in the y direction toward the feet, indicating that the subject is inhaling.)

The absolute direction values of the pixel in the image of the region-of-interest ROI, which have been obtained by the method described above, may be added together. Then, the changes in respiration volume (either expiration volume or aspiration volume) can be detected.

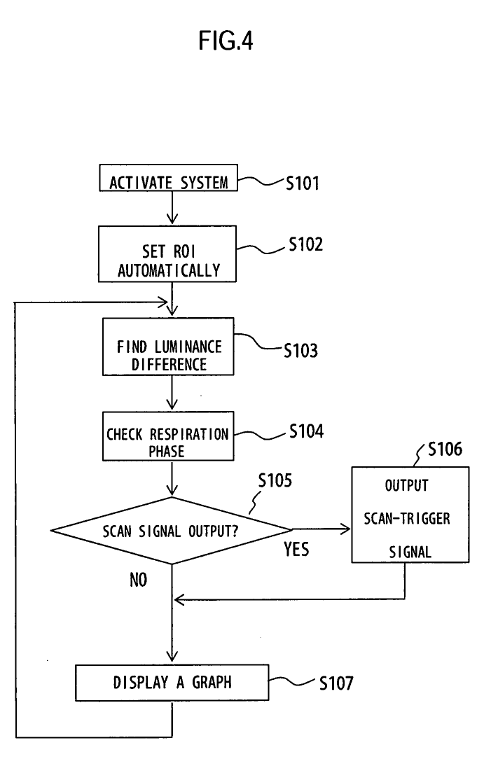

FIG. 4 is a flowchart that explains the entire sequence of a respiration monitoring method performed in the embodiment of this invention.

First, the system is activated (S101).

Next, the imaging unit 101 makes an initial setting, or designates a region ROI to be photographed (i.e., so-called region-of-interest) that includes the chest or abdomen of the subject M (S102). The region thus designated is, for example, a region in which the luminance change is most prominent as determined from the difference between the consecutive frames in terms of the luminance of the pixel.

Then, the respiration-cycle determining unit 102 and the expiration/aspiration discriminating unit 103 perform a difference process, finding the difference in luminance between the pixels forming the image acquired (S103). On the basis of the result of the difference process, the respiration cycle determined and the results of the expiration/aspiration discrimination, the expiration/aspiration timing determining unit 104 determines the timing of expiration and the timing of aspiration.

The scan-signal output unit 2 then checks the phase of the respiration-waveform cycle (S104). Then, the unit 2 determines whether the timing of expiration or aspiration is a timing of outputting the scan signal. If the timing is a timing of outputting the scan signal (that is, if YES in S105), the scan-signal output unit 2 outputs the scan signal to the CT-scan apparatus 3 (S106).

Thereafter, the display unit (not shown) displays a graph (S107). The graph represents the respiration cycle and the expiration/aspiration timing that have been determined by the respiration-cycle determining unit 102, expiration/aspiration discriminating unit 103 and expiration/aspiration timing determining unit 104. The display unit displays a graph showing a standard respiration model, together with the graph that shows the respiration cycle and the expiration/aspiration timing. By comparing the two graphs simultaneously displayed, the user can easily determine whether the subject's respiration is normal or not.

The timing may not be a timing of outputting the scan signal (that is, if NO in S105), the display unit (not shown) displays a graph showing this fact (S107).

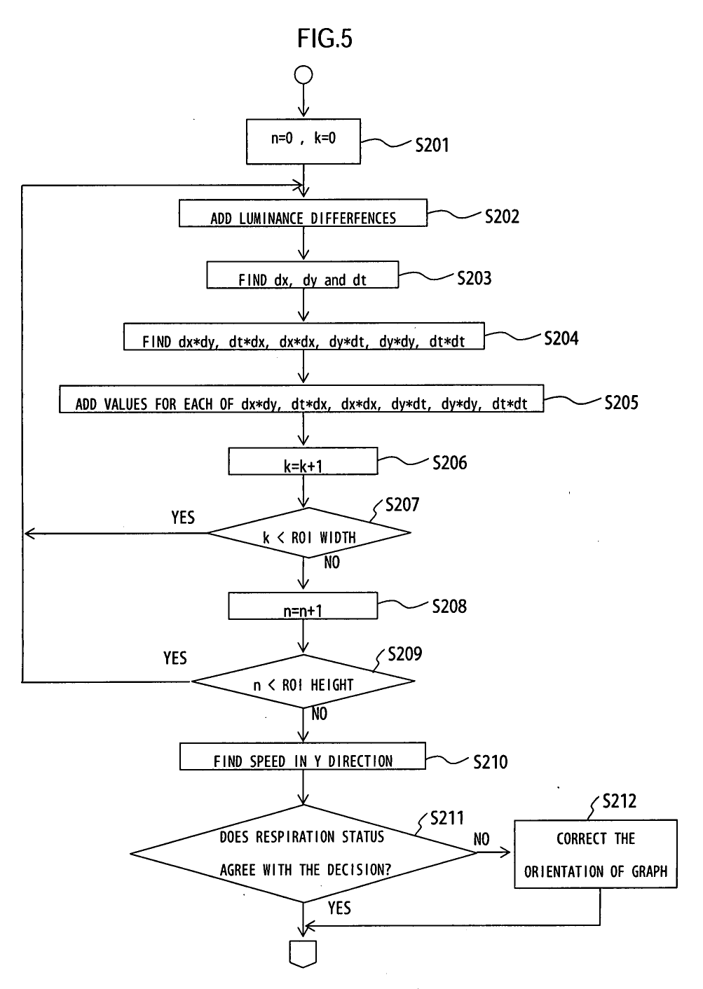

FIG. 5 is a flowchart explaining, in detail, the difference process (S103) shown in FIG. 4.

At first, the indices n and k of the pixel in the image are initialized (S201).

Next, the image-acquiring unit 105 performs a difference process on the between the luminance values the pixel (designated by the indices) has in several images obtained at consecutive timings (S202). (The difference process is a process of adding the absolute values of the luminance differences, each being pixel-luminance difference between two adjacent frames.)

Next, the luminance difference dx between the pixels arranged in the X direction, the luminance difference dy between the pixels arranged in the Y direction, and the luminance difference dt between the frames obtained at different timings for the pixel at the same position are calculated (S203).

Then, from dx, dy and dt obtained in Step S203, dxXdy, dtXdx, dxXdx, dyXdt, dyXdy and dtXdt are obtained (S204).

The results attained in Step S204 are added for each item involved in Step S204 (S205).

The X-direction index k of the pixel is incremented by 1 (S206). It is then determined whether the X-direction index k has yet to exceed the width of the regions-of-interest ROI (S207).

If the X-direction index k has exceeded the width of the regions-of-interest ROI, as measured in the X direction (that is, if NO in Step S207), the Y-direction index n of the pixel is incremented by 1 (S208).

It is then determined whether the Y-direction index n has yet to exceed the height of the regions-of-interest ROI, as measured in the Y direction (S209).

Thus, the difference process is performed on the luminance of the pixel as long as the indices of the pixel remain in the range of the region-of-interest ROI.

Using the equation (1), the Y-direction positional change with time (change in speed and orientation) is calculated for all pixels in the region ROI that should be photographed (S210).

On the basis of the Y-direction positional change with time, calculated for all pixels in Step S210, it is determined whether the pixel in the region ROI that should be photographed is moving toward the head or feet (S211). At this point, it is also determined whether the status of the present respiration agrees with the results of Step S211.

Assume that the status of the present respiration is aspiration, and that the pixel in the region ROI that should be photographed is moving toward the head (moving in the first direction). Then, the operation goes to the process (S201) on the pixel in the next frame (i.e., the image frame acquired at the timing next to the timing at which the frame being processed now has been acquired).

On the other hand, the status of the present respiration may be aspiration, and the pixel in the region ROI that should be photographed may be moving toward the feet (moving in the second direction). In this case, the status of the present respiration does not agree with the results of Step S211. Hence, the results of discrimination between expiration and aspiration are corrected to “expiration” (S212).

Thus, the data (containing the data item about the respiration status) obtained by the expiration/aspiration discriminating unit 103, respiration-cycle determining unit 102 and expiration/aspiration timing determining unit 104 is stored in the storage unit (not shown).

### Second Embodiment

The second embodiment of the present invention will be described.

This embodiment is a modification of the first embodiment. Therefore, the components identical with those of the first embodiment are designated by the same reference numbers and will not be described. The present embodiment differs from the first embodiment in that the expiration/aspiration discriminating unit 103 determines, by a different method, the direction the pixel moves in the image photographed by the imaging unit 101.

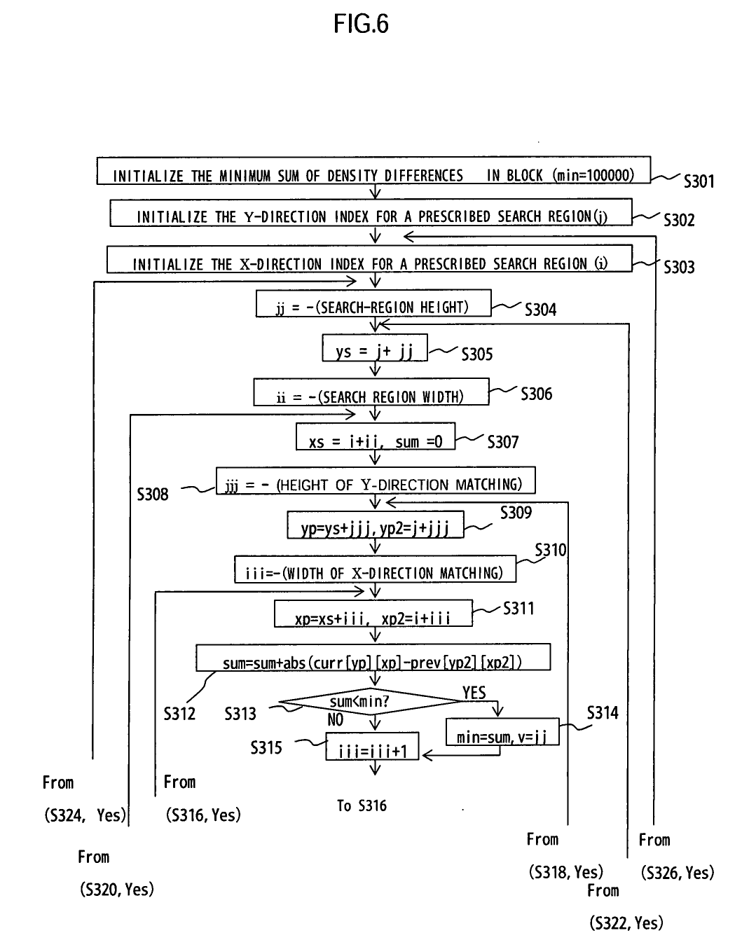

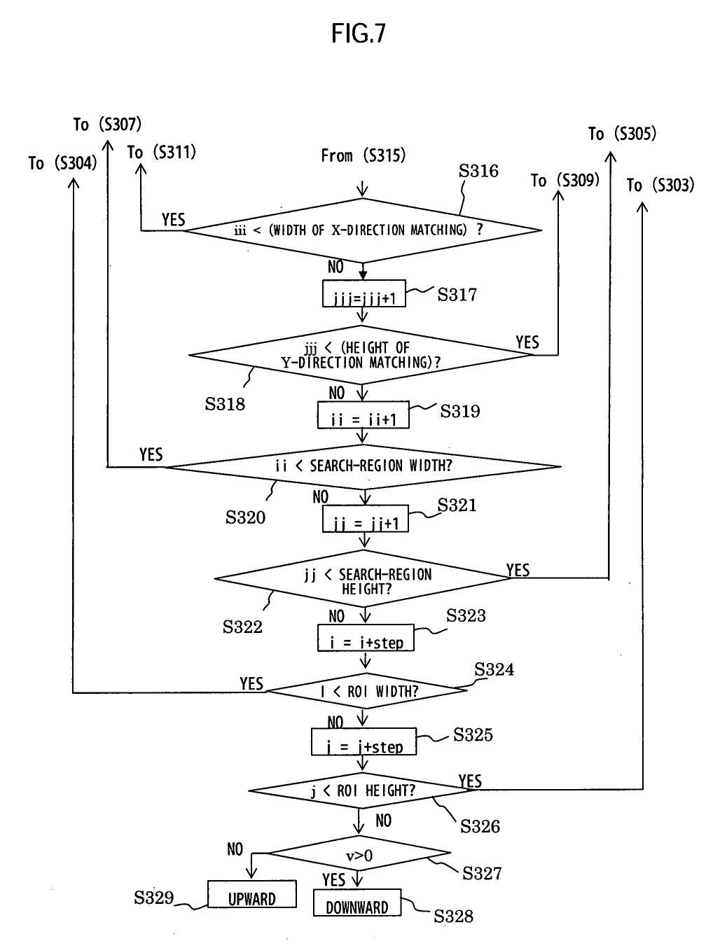

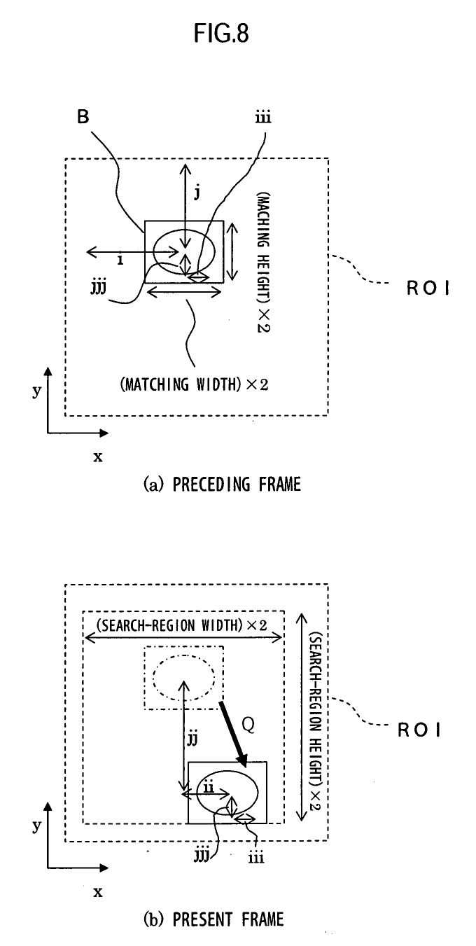

FIGS. 6 and 7 are flowcharts explaining a method of determining how a pixel moves in the image, by using the expiration/aspiration discriminating unit in the present embodiment. Note that a single flowchart has been divided into FIGS. 6 and 7, for the sake of convenience. FIG. 8 is a diagram showing the motion of a specific block and explaining a method of matching the block. This figure shows that at the timing of the current frame (b), block B in the immediately preceding frame (a) has moved in the direction of arrow Q to a specific position.

Expiration and aspiration may be discriminated, one from the other, as follows. Consider an image of the region-of-interest ROI, acquired at a certain timing (present frame), and an image of the region-of-interest ROI, acquired at the immediately preceding timing (previous frame). A plurality of rectangular blocks B (first region) are set in the region-of-interest ROI, existing in the present frame. Also, a plurality of rectangular blocks B (first region) are set in the region-of-interest ROI, existing in the previous frame. Note that the rectangular blocks B are of the same size, obtained by dividing the region ROI in both the X direction and the Y direction. Each rectangular block of the previous frame is compared with the corresponding rectangular block of the present frame, in term of the density of each pixel. Then, the differences in pixel density are added for every block.

At this time, the blocks of the previous frame are compared with those of the present frame in terms of pixel distribution, by moving each block B of the previous frame in the region where the corresponding block B of the present frame existed in the previous block B, unit by unit smaller than the block B. Thus, the accurate matching can be achieved even if the block B moves minutely. The matching can, of course, be performed in units of small rectangular blocks obtained by dividing the region ROI as described above.

The results of adding the differences in pixel density, obtained for all blocks of the previous frame, are stored in the storage unit (not shown). All blocks of the previous frames are then added together.

As a result, a block of previous frame and a block (second region) of the present frame may be found, between which the density difference is the smallest. Then, these two blocks are considered to be most similar to each other than any other pair of blocks compared, in terms of pixel pattern (i.e., pattern defined by pixels). From this it can be inferred that the block of the previous frame has moved to the position where the corresponding block of the present frame has assumed previously.

Thus, it is inferred that the object of photography (i.e., object to examine) existing in the region-of-interest ROI has moved for the distance the block of the previous frame has so moved as described above. The distance and direction the block has moved is regarded as a vector extending from a point in the block of the previous frame to the identical point in the block of the current frame. Whether the object is oriented upward or downward is determined in accordance which sign, positive or negative, the Y-direction component of the vector.

First, a buffer for storing the minimum sum of the density differences in a block set in the region-of-interest ROI is initialized (S301). In the algorithm applied in the present embodiment, the numerical value to substitute for “min” should preferably be as great as possible.

A prescribed search region is set in the Y direction, and the Y-direction index j is initialized (S302).

Next, a prescribed search region is set in the X direction, and the X-direction index i is initialized (S303).

Subsequently, the index for the height of the search region (i.e., index for setting the search region in the Y direction) is initialized (S304).

The results of calculation for enhancing the process are stored in a buffer (S305).

The index of the width of the search region (i.e., index for setting the search region in the X direction) is initialized (S306).

The results of calculation for enhancing the process are stored in the buffer, and the buffer for storing the sum of intra-block density-difference is initialized (S307).

The index of the matching height (negative value that is half (½) the dimension a specific block B has as measured in the Y direction) is initialized (S308).

The results of calculation for enhancing the process are stored in the buffer (S309).

The index of the matching width (negative value that is half (½) the dimension the specific block B has as measured in the X direction) is initialized (S310).

The results of calculation for enhancing the process are stored in the buffer (S311).

The sums of the absolute values of density differences (differences in luminance between the pixels) in the specific block B are added together (S312).

The minimum sum of the density differences in the specific block B is compared with the sum of the intra-block density-differences (S313).

The sum of the intra-block density-differences is stored in the buffer that stores the minimum sum of the density differences between pixels in the specific block B (S314).

Next, the index of the matching width is incremented (S315).

It is determined whether the index of the matching width is smaller than the matching width measured in the X direction (S316).

Then, the index of the matching height is incremented (S317).

Further, it is determined whether the index of the matching height is smaller than the matching height measured in the Y direction (S318).

The index of the width of the search region is incremented (S319).

It is then determined whether the index of the width of the search region is smaller than the width of the search region (S320).

The index of the height of the search region is incremented (S321).

Next, it is determined whether the index of the height of the search region is smaller than the height of the search region (S322).

The search region is determined, and the index in the X direction is incremented (S323).

The search region is determined, and it is determined whether the index in the X direction is smaller than the width of the region-of-interest ROI (S324).

The search region is determined, and the index in the Y direction is incremented (S325).

The search region is determined, and it is determined whether the index in the Y direction is smaller than the height of the region-of-interest ROI (S326).

Further, the direction, in which the image changes (with time) in the region-of-interest ROI, is determined (S327). It is thus determined in which direction the pixel moves, upwards or downward in the region-of-interest ROI (S329 and S328).

That is, in this embodiment, the expiration/aspiration discriminating unit discriminates between expiration and aspiration in the following way. The second region of an image acquired at a prescribed timing and having pixels of almost the same luminance distribution as those of the first region composed of given pixels existing in the image acquired at the timing immediately preceding that prescribed timing is extracted. If the component of the direction in which the pixel moves from the first region to the second region is oriented toward the subject's head, it is determined that the subject is inhaling. If that component of the direction is oriented toward the subject's feet, it is determined that the subject is exhaling.

In the first and second embodiments, the imaging unit 101 is located above the feet of the subject M lying on the back and photographs the region-of-interest ROI in a direction slantwise with respect to the region-of-interest ROI. The present invention is not limited to this configuration. For example, the imaging unit 101 may be located above the head of the subject M lying on the back and photographs the region-of-interest ROI in a direction slantwise with respect to the region-of-interest ROI. If this is the case, it is determined that the subject M is inhaling if the pixels move in the image toward the feet of the subject M (in the first direction) and that the subject M is exhaling if the pixels move toward the head of the subject M (in the second direction). The imaging unit 101 can of course be so positioned to photograph the region-of-interest ROI existing in the subject's side, from above in a direction slantwise to the region-of-interest ROI. In other words, the embodiments can have any configuration so long as the unit 101 can photograph the region-of-interest ROI, slantwise with respect of the chest or abdomen of the subject M.

### Third Embodiment

The third embodiment of the present invention will be described below.

This embodiment is a modification of the first embodiment. Therefore, the components identical with those of the first embodiment are designated by the same reference numbers and will not be described. The present embodiment differs from the first embodiment in that the expiration/aspiration discriminating unit determines, by a different method, discriminate between expiration and aspiration.

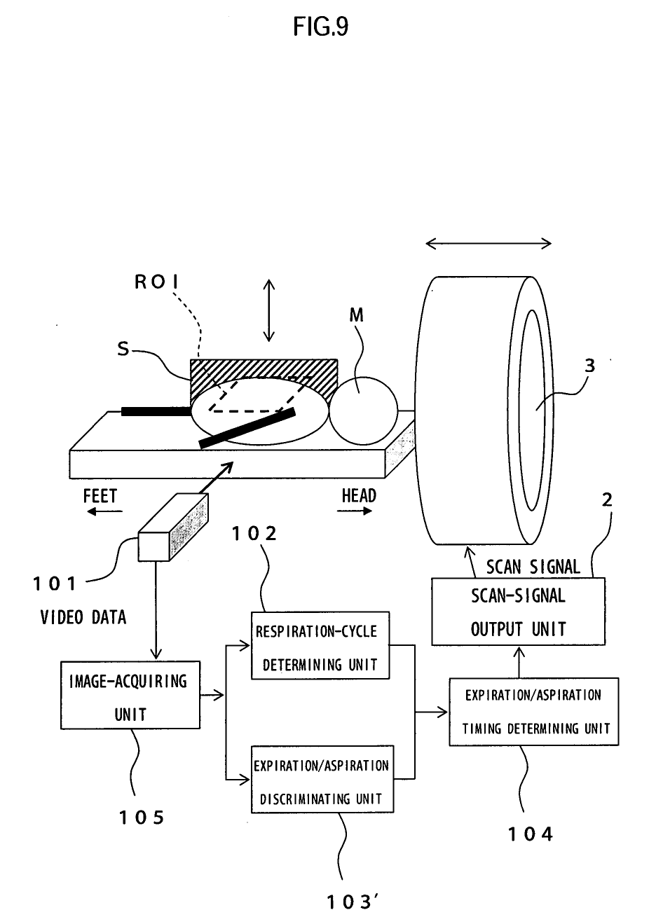

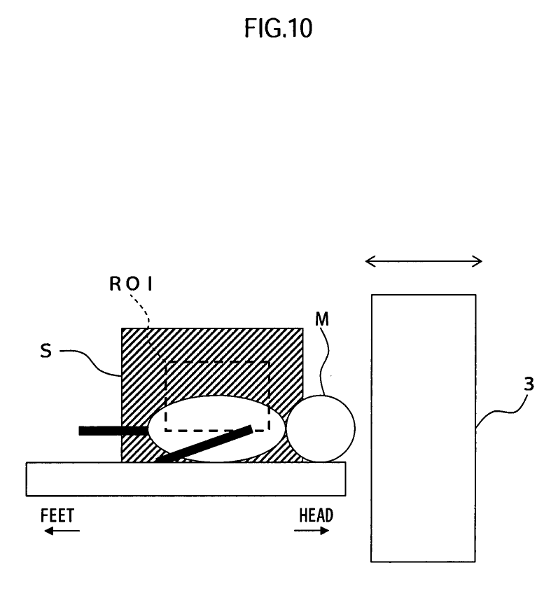

In the first embodiment, the imaging unit 101 is arranged, inclining to the region-of-interest ROI, which exists in the subject (see FIGS. 1 and 2). In the present embodiment, as shown in FIGS. 9 and 10, the imaging unit 101 is arranged to photograph the region-of-interest ROI, which includes the chest or abdomen of the subject M, or both, sideways with respect to the subject M. The region-of-interest ROI also includes the boundary between the chest or abdomen of the subject M, or both, and the background S. The backgrounds in the region-of-interest ROI has lower illuminance than the chest or abdomen, or both.

As the subject M inhales, the abdomen or chest of the subject M swells. Conversely, the abdomen or chest shrinks as the subject M exhales. Hence, that part of the image of the region-of-interest ROI, which has high illuminance, expands as the subject M inhales, and contracts as the subject M exhales, as can be seen from the change in the luminance difference between the pixels in the image of the region-of-interest ROI. Expiration and aspiration can therefore be discriminated, one from the other.

That is, the expiration/aspiration discriminating unit 103′ discriminates between expiration and aspiration, on the basis of a plurality of images the image-acquiring unit has acquired at consecutive timings and in accordance with the change in area of that part of image, which is defined by pixels having luminance higher than a predetermined value in the region-of-interest. More precisely, the unit 103′ determines that the subject is inhaling if the area of the high-illuminance part in the region-of-interest increases, and that the subject is exhaling if the area of the high-illuminance part decreases.

To achieve an effective suppression of motion artifacts developing from respiration in the imaging apparatus such as CT-scan apparatus 3, it is desired that the maximum respiration volume, i.e., the amplitude of respiration, be constant at all times.

In view of this, the absolute values of all inter-frame, pixel-density differences in the region-of-interest ROI are added while the subject is inhaling. It is then determined whether the maximum value thereby obtained (i.e., the maximum depth of respiration) remains constant at all times. If the maximum value changes, this fact should preferably be notified in the form of, for example, an audio message.

More specifically, the sum of the absolute values of inter-frame, pixel-density (luminance) differences in the region-of-interest ROI, which have been obtained during a standard respiration model is added during the aspiration, and is subtracted during the expiration. The results of the addition or subtraction are stored in time sequence. The timings of expiration or aspiration, thus stored, are notified to the subject in the form of audio messages, thus prompting the subject to breath in synchronism with the messages. Then, it is determined whether the maximum depth of the subject's respiration is similar to the maximum depth of the standard respiration model. If the respiration depth or cycle differs from the standard respiration depth or standard respiration cycle by a value greater than the prescribed value, this fact will be notified by a notification unit (not shown).

That is, the absolute values of the luminance changes of the pixels in the images acquired by the image-acquiring unit at predetermined consecutive timings are accumulated as long as the expiration/aspiration discriminating unit determines that the subject is inhaling. When the difference between the value thus accumulated and a predetermined value exceeds a preset value, the notification unit (not shown) notifies that the subject is inhaling.

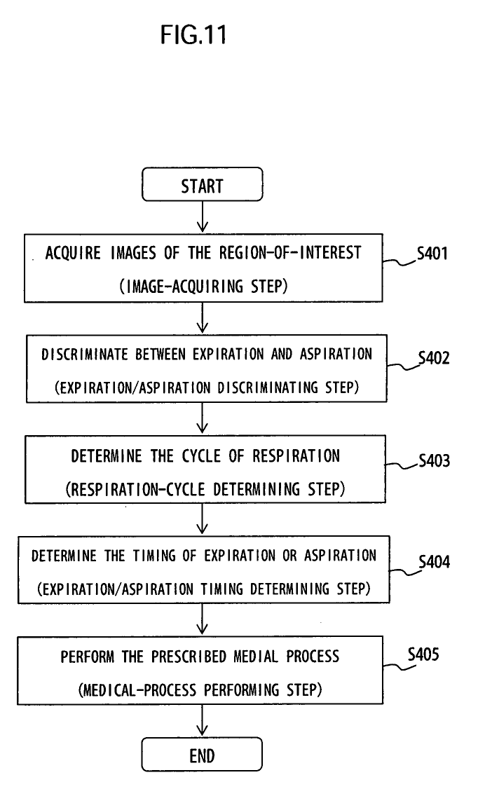

FIG. 11 is a flowchart showing the entire sequence of the respiration monitoring method performed by using a medical processing system that includes a respiration monitoring apparatus according to the present embodiment.

First, images of the region-of-interest including the chest or abdomen of the subject, or both, are acquired at predetermined timings by photographing the region-of-interest at a predetermined angle (image-acquiring step S401).

On the basis of the images acquired at the consecutive predetermined timings in the image-acquiring step, the direction in which the pixels move is determined from the positional changes the pixels in the images undergo as time passes. Based on the direction thus determined, the subject's expiration and aspiration are discriminated, one from the other (expiration/aspiration discriminating step) (S402).

On the basis of the images acquired at the consecutive predetermined timings in the image-acquiring step, too, the cycle of the subject's respiration is determined from the luminance changes the pixels in the images undergo as time passes (respiration-cycle determining step) (S403).

Subsequently, on the basis of the respiration cycle determined in the respiration-cycle determining step, the timing of either the expiration or aspiration, which has been determined in the expiration/aspiration discriminating step, is determined (expiration/aspiration timing determining step) (S404).

Next, the scan-signal output unit 2 outputs a scan signal at the expiration or aspiration timing determined in the expiration/aspiration timing determining step, whereby a CT scan, i.e., the prescribed medial process, is performed (medical-process executing step) (S405).

The control unit (not shown) executes a respiration monitoring program stored in the storage unit (not shown). The steps of the respiration monitoring method described above are thereby performed.

In the present embodiment, the programs describing the functions of this invention are stored in the apparatus as described above. Alternatively, the programs may be downloaded into the apparatus via a network, or a recording medium storing the programs may be installed in the apparatus. The recording medium may be of any type, such as a CD-ROM, so long as it can store the programs and the programs can be read from it. The functions that can be implemented once installed or downloading into the apparatus may of the type that cooperates with the operating system (OS) installed in the apparatus.

In this embodiment, the respiration is detected to achieve a respiration-synchronized medical process, in a particular method. That is, the imaging unit is located above the subject's feet and photographs the subject's chest or abdomen from above, in a direction slantwise with respect to the chest or abdomen, thereby finding any part that is moving. This part is regarded as region-of-interest ROI. The difference between the images of the part, acquired at consecutive timings, is detected as a change in respiration. The absolute value of this change is used as data for emphasizing the change.

In the embodiments described above, the expiration/aspiration discriminating unit 103 discriminates between expiration and aspiration, before the respiration-cycle determining unit 102 determines the cycle of respiration. The invention is not limited to this configuration. The respiration-cycle determining unit 102 and the expiration/aspiration discriminating unit 103 may operate in whichever order possible. Instead, these units 102 and 103 may operate at the same time.

In the present embodiment, the medical process that the medical-process executing unit performs is, for example, a CT scan carried out by using a CT-scan apparatus. The medical process is not limited to this, nevertheless. It may be any other tomography using, for example, a magnetic-resonance imaging (MRI) apparatus, or a surgical treatment.

The present invention has been described, with reference to specific embodiments. Nevertheless, it will be obvious to those skilled in the art that various changes and modifications can be made without departing from the scope and spirit of the present invention.

### INDUSTRIAL APPLICABILITY

As has been described, the present invention can provide a technique that can discriminate between the subject's expiration and aspiration, without contacting the subject.

1. A respiration monitoring apparatus comprising:
an image-acquiring unit that acquires images at predetermined timings, each image photographed in a direction inclined at a prescribed angle to a region-of-interest including at least one of a subject's chest and abdomen; and
an expiration/aspiration discriminating unit that determines a direction in which a pixel moves, from a positional change of the pixel in an image, on the basis of a plurality of images acquired at consecutive timings by the image-acquiring unit, and discriminates between the subject's expiration and aspiration on the basis of the direction determined.

2. The respiration monitoring apparatus according to claim 1, wherein the expiration/aspiration discriminating unit determines that the subject is inhaling when the pixel in the image moves in a first direction that is a direction component in a plane substantially parallel to longitudinal and transverse directions of the subject, and that the subject is exhaling when the pixel in the image moves in a second direction that is opposite to the first direction.

3. The respiration monitoring apparatus according to claim 2, wherein a speed dy/dt of all pixels in the region-of-interest is given as follows with respect to the direction substantially parallel to the longitudinal direction of the subject:
$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$
where y is an ordinate on the image acquired by the image-acquiring unit with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y) at the time t, the first E: is the sum for all pixels with respect to one of the x and y directions in the region-of-interest, and the second E is the sum for all pixels with respect to the other of the x and y directions in the region-of-interest, and
the expiration/aspiration discriminating unit determines that the subject is inhaling when the pixel moves at the speed dy/dt in the first direction and that the subject is exhaling when the pixel moves at speed dy/dt in the second direction.

4. The respiration monitoring apparatus according to claim 2, wherein the expiration/aspiration discriminating unit extracts a second region of an image acquired at a prescribed timing and having pixels of almost the same luminance distribution as those of a first region composed of given pixels existing in the image acquired at the timing immediately preceding that prescribed timing, and determines that the subject is inhaling when the component of the direction in which the pixel moves from the first region to the second region is oriented in the first direction and that the subject is exhaling when the component of the direction is oriented in the second direction.

5. The respiration monitoring apparatus according to claim 1, further comprising:
a respiration-cycle determining unit that determines a respiration cycle of the subject, on the basis of images acquired at the predetermined consecutive timings by the image-acquiring unit and from the change in the luminance of pixels in the images, which occurs with time; and
an expiration/aspiration timing determining unit that determines the timing of the expiration or aspiration discriminated by the expiration/aspiration discriminating unit, on the basis of the respiration cycle determined by the respiration-cycle determining unit.

6. A respiration monitoring apparatus comprising:
an image-acquiring unit that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and
an expiration/aspiration discriminating unit that discriminates between the subject's expiration and aspiration, on the basis of a plurality of images acquired at the predetermined timings by the image-acquiring unit and in accordance with whether a part of the region-of-interest, which is composed of pixels having luminance higher than a prescribed value, increases or decreases in area.

7. The respiration monitoring apparatus according to claim 6, wherein the expiration/aspiration discriminating unit determines that the subject is inhaling when that part of the region-of-interest, which is composed of pixels having luminance higher than a prescribed value, increases in area, and that subject is exhaling when that part of the region-of-interest, which is composed of pixels having luminance higher than a prescribed value, decreases in area.

8. The respiration monitoring apparatus according to claim 6, further comprising:
a respiration-cycle determining unit that determines a respiration cycle of the subject, on the basis of images acquired at the predetermined consecutive timings by the image-acquiring unit and from the change in the luminance of pixels in the images, which occurs with time; and
an expiration/aspiration timing determining unit that determines the timing of the expiration or aspiration discriminated by the expiration/aspiration discriminating unit, on the basis of the respiration cycle determined by the respiration-cycle determining unit.

9. The respiration monitoring apparatus according to claim 5, further comprising a notification unit that accumulates the absolute values of the luminance changes of the pixels in the images acquired by the image-acquiring unit at predetermined consecutive timings, as long as the expiration/aspiration discriminating unit determines that the subject is inhaling, and notifies that the subject is inhaling, when a difference between the value thus accumulated and a predetermined value exceeds a preset value.

10. A respiration monitoring system comprising:
a respiration monitoring apparatus of such a type as described in claim 1; and
an imaging unit that is located above the feet of the subject lying on the back and photographs the region-of-interest in a direction slantwise with respect to the region-of-interest.

11. A respiration monitoring system comprising.:
a respiration monitoring apparatus of such a type as described in claim 6; and
an imaging unit that photographs, sideways with respect to a subject, a region-of-interest including at least one of the subject's chest and abdomen and a boundary between at least one of the chest and abdomen and a background having lower illuminance than at least one of the chest and abdomen.

12. A medical processing system having:
a respiration monitoring apparatus of such a type as described in claim 5; and
a medical-process executing unit that performs a predetermined medical process at a timing of the expiration or aspiration determined by the expiration/aspiration timing determining unit.

13. The medical processing system according to claim 12, wherein the predetermined medical process is an imaging process performed by MRI.

14. The medical processing system according to claim 12, wherein the predetermined medical process is an imaging process performed by CT scan.

15. A respiration monitoring method comprising:
an image-acquiring step that acquires images at predetermined timings, each image photographed in a direction inclined at a prescribed angle to a region-of-interest including at least one of a subject's chest and abdomen; and
an expiration/aspiration discriminating step that determines a direction in which a pixel moves, from a positional change of the pixel in an image, on the basis of a plurality of images acquired at consecutive timings in the image-acquiring step, and discriminates between the subject's expiration and aspiration on the basis of the direction determined.

16. The respiration monitoring method according to claim 15, wherein in the expiration/aspiration discriminating step, the subject is determined to be inhaling when the pixel in the image moves in a first direction that is a direction component in a plane substantially parallel to longitudinal and transverse directions of the subject, and is determined to be exhaling when the pixel in the image moves in a second direction that is opposite to the first direction.

17. The respiration monitoring method according to claim 16, wherein a speed dy/dt of all pixels in the region-of-interest is given as follows with respect to the direction substantially parallel to the longitudinal direction of the subject:
$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$
where y is an ordinate on the image acquired by the image-acquiring step with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y), the first Σ is the sum for all pixels with respect to one of the x and y directions in the region-of-interest, and the second Σ is the sum for all pixels with respect to the other of the x and y directions in the region-of-interest, and
in the expiration/aspiration discriminating step, the subject is determined to be inhaling when the pixel moves at the speed dy/dt in the first direction and is determined to be exhaling when the pixel moves at speed dy/dt in the second direction.

18. The respiration monitoring method according to claim 16, wherein in the expiration/aspiration discriminating step, a second region of an image acquired at a prescribed timing and having pixels of almost the same luminance distribution as those of a first region composed of given pixels existing in the image acquired at the timing immediately preceding that prescribed timing is extracted, and the subject is determined to be inhaling when the component of the direction in which the pixel moves from the first region to the second region is oriented in the first direction and to be exhaling when the component of the direction is oriented in the second direction.

19. The respiration monitoring method according to claim 15, further comprising:
a respiration-cycle determining step that determines a respiration cycle of the subject, on the basis of images acquired at the predetermined consecutive timings in the image-acquiring step and from the change in the luminance of pixels in the images, which occurs with time; and
an expiration/aspiration timing determining step that determines the timing of the expiration or aspiration discriminated in the expiration/aspiration discriminating step, on the basis of the respiration cycle determined in the respiration-cycle determining step.

20. A respiration monitoring method comprising:
an image-acquiring step that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and
an expiration/aspiration discriminating step that discriminates between the subject's expiration and aspiration, on the basis of a plurality of images acquired at the predetermined timings in the image-acquiring step and in accordance with whether a part of the region-of-interest, which is composed of pixels having luminance higher than a prescribed value, increases or decreases in area.

21. The respiration monitoring method according to claim 20, wherein in the expiration/aspiration discriminating step, the subject is determined to be inhaling when that part of the region-of-interest, which is composed of pixels having luminance higher than a prescribed value, increases in area, and the subject is determined to be exhaling when that part of the region-of-interest, which is composed of pixels having luminance higher than a prescribed value, decreases in area.

22. The respiration monitoring method according to claim 20, further comprising:
a respiration-cycle determining step that determines a respiration cycle of the subject, on the basis of images acquired at the predetermined consecutive timings in the image-acquiring step and from the change in the luminance of pixels in the images, which occurs with time; and
an expiration/aspiration timing determining step that determines the timing of the expiration or aspiration discriminated in the expiration/aspiration discriminating step, on the basis of the respiration cycle determined in the respiration-cycle determining step.

23. The respiration monitoring method according to claim 19, further having a notification step that accumulates the absolute values of the luminance changes of the pixels in the images acquired in the image-acquiring step at predetermined consecutive timings, as long as the subject is determined to be inhaling in the expiration/aspiration discriminating step, and notifies that the subject is inhaling, when a difference between the value thus accumulated and a predetermined value exceeds a preset value.

24. The respiration monitoring method according to claim 19, having a medical-process executing step that performs a predetermined medical process at a timing of the expiration or aspiration determined in the expiration/aspiration timing determining step.

25. The respiration monitoring method according to claim 24, wherein the predetermined medical process is an imaging process performed by MRI.

26. The respiration monitoring method according to claim 24, wherein the predetermined medical process is an imaging process performed by CT scan.

27. A respiration monitoring program enabling a computer to perform:
an image-acquiring step that acquires images at predetermined timings, each image photographed in a direction inclined at a prescribed angle to a region-of-interest including at least one of a subject's chest and abdomen; and
an expiration/aspiration discriminating step that determines a direction in which a pixel moves, from a positional change of the pixel in an image, on the basis of a plurality of images acquired at consecutive timings in the image-acquiring step, and discriminates between the subject's expiration and aspiration on the basis of the direction determined.

28. The respiration monitoring program according to claim 27, wherein in the expiration/aspiration discriminating step, the subject is determined to be inhaling when the pixel in the image moves in a first direction that is a direction component in a plane substantially parallel to longitudinal and transverse directions of the subject, and is determined to be exhaling when the pixel in the image moves in a second direction that is opposite to the first direction.

29. The respiration monitoring program according to claim 28, wherein a speed dy/dt of all pixels in the region-of-interest is given as follows with respect to the direction substantially parallel to the longitudinal direction of the subject:
$$\begin{matrix}\frac{d y}{d t} = - ( - \sum \sum ( ( \partial I ( x , y , t ) / \partial x ) * ( \partial I ( x , y , t ) / \partial y ) ) * \\ \sum \sum ( ( \partial I ( x , y , t ) / \partial t ) * ( \partial I ( x , y , t ) / \partial y ) ) + \\ \sum \sum ( {( \partial I ( x , y , t ) / \partial x )}^{2} * \\ \sum \sum ( ( \partial I ( x , y , t ) / \partial y ) * ( \partial I ( x , y , t ) / \partial t ) ) ) / \\ ( \sum \sum ( {( \partial I ( x , y , t ) / \partial y )}^{2} * \sum \sum ( {( \partial I ( x , y , t ) / \partial x )}^{2} - \\ ( \sum \sum {( ( \partial I ( x , y , t ) / \partial x ) * ( \partial I ( x , y , t ) / \partial y ) )}^{2} )\end{matrix}$$
where y is an ordinate on the image acquired by the image-acquiring step with respect to the longitudinal direction of the subject, x is an abscissa on the image with respect to the direction intersecting at substantially right angles with the longitudinal direction of the subject, t is time, I(x,y,t) is the luminance of the pixel positioned at the coordinate (x,y) at the time t, the first Σ is the sum for all pixels with respect to one of the x and y directions in the region-of-interest, and the second Σ is the sum for all pixels with respect to the other of the x and y directions in the region-of-interest, and
in the expiration/aspiration discriminating step, the subject is determined to be inhaling when the pixel moves at the speed dy/dt in the first direction and is determined to be exhaling when the pixel moves at speed dy/dt in the second direction.

30. The respiration monitoring program according to claim 28, wherein, in the expiration/aspiration discriminating step, a second region of an image acquired at a prescribed timing and having pixels of almost the same luminance distribution as those of a first region composed of given pixels existing in the image acquired at the timing immediately preceding that prescribed timing is extracted, and the subject is determined to be inhaling when the component of the direction in which the pixel moves from the first region to the second region is oriented in the first direction and to be exhaling when the component of the direction is oriented in the second direction.

31. The respiration monitoring program according to claim 27, further having:
a respiration-cycle determining step that determines a respiration cycle of the subject, on the basis of images acquired at the predetermined consecutive timings in the image-acquiring step and from the change in the luminance of pixels in the images, which occurs with time; and
an expiration/aspiration timing discriminating step that determines the timing of the expiration or aspiration discriminated in the expiration/aspiration discriminating step, on the basis of the respiration cycle determined in the respiration-cycle determining step.

32. A respiration monitoring program enabling a computer to perform:
an image-acquiring step that acquires images at predetermined timings by photographing, sideways with respect to a subject, a region-of-interest including at least one of the subject's chest and abdomen and a boundary between at least one of the chest and abdomen and a background having lower illuminance than at least one of the chest and abdomen; and
an expiration/aspiration discriminating step that discriminates between the subject's expiration and aspiration, on the basis of a plurality of images acquired at the predetermined timings in the image-acquiring step and in accordance with whether a part of the region-of-interest, which is composed of pixels having luminance higher than a prescribed value, increases or decreases in area.

33. The respiration monitoring program according to claim 32, wherein in the expiration/aspiration discriminating step, the subject is determined to be inhaling when that part of the region-of-interest, which is composed of pixels having luminance higher than a prescribed value, increases in area, and the subject is determined to be exhaling when that part of the region-of-interest, which is composed of pixels having luminance higher than a prescribed value, decreases in area.

34. The respiration monitoring program according to claim 32, further comprising:
a respiration-cycle determining step that determines a respiration cycle of the subject, on the basis of images acquired at the predetermined consecutive timings in the image-acquiring step and from the change in the luminance of pixels in the images, which occurs with time; and
an expiration/aspiration timing determining step that determines the timing of the expiration or aspiration discriminated in the expiration/aspiration discriminating step, on the basis of the respiration cycle determined in the respiration-cycle determining step.

35. The respiration monitoring program according to claim 31, further having a notification step that accumulates the absolute values of the luminance changes of the pixels in the images acquired in the image-acquiring step at predetermined consecutive timings, as long as the subject is determined to be inhaling in the expiration/aspiration discriminating step, and notifies that the subject is inhaling, when a difference between the value thus accumulated and a predetermined value exceeds a preset value.

36. The respiration monitoring program according to claim 31, having a medical-process executing step that performs a predetermined medical process at a timing of the expiration or aspiration determined in the expiration/aspiration timing determining step.

37. The respiration monitoring program according to claim 36, wherein the predetermined medical process is an imaging process performed by MRI.

38. The respiration monitoring program according to claim 36, wherein the predetermined medical process is an imaging process performed by CT scan.
